(19) United States
(12) Patent Application Publication
(10) Pub. No.: US 2025/0248617 A1
(43) Pub. Date: Aug. 7, 2025

(54) AUTOMATIC CARDIOVASCULAR MAGNETIC RESONANCE IMAGING

(71) Applicant: The Cleveland Clinic Foundation, Cleveland, OH (US)

(72) Inventors: Christopher T. NGUYEN, Beachwood, OH (US); David CHEN, Independence, OH (US); Deborah KWON, Cleveland Heights, OH (US); Danielle Kara, Cleveland, OH (US)

(21) Appl. No.: 19/036,237

(22) Filed: Jan. 24, 2025

Related U.S. Application Data
(60) Provisional application No. 63/550,802, filed on Feb. 7, 2024.

(51) Int. Cl. A61B 5/055; A61B 5/00

(52) U.S. Cl. CPC A61B 5/055; A61B 5/0044

(57) ABSTRACT

The present disclosure relates to automated cardiovascular magnetic resonance imaging systems and methods that can be used to produce both cine and delayed enhancement images with minimal interaction by a clinician and minimal dependence on system hardware and clinician skill and experience. The images may be acquired during patient free-breathing and does not require a contrast agent. The resulting images be time-resolved three-dimensional images having isotropic resolution, permitting retrospective two-dimensional image reconstruction in any plane image reconstruction according to a retrospectively determined inversion time.

### CROSS-REFERENCE TO RELATED APPLICATIONS

This application claims priority to U.S. Provisional Patent Application No. 63/550,802 filed Feb. 7, 2024, the entirety of which is incorporated by reference herein in its entirety.

### BACKGROUND

Cardiovascular Magnetic Resonance (CMR) Imaging, also referred to as “Cardiac MRI”, is used to assess structure, function, flow, and physiology of the cardiovascular system by utilizing specific acquisition parameters (e.g., ECG gating, scan protocols) to visually characterize cardiac tissue. While CMR remains a useful tool, conventional CMR is slow (a single scan may take 90 minutes for setup, localization, acquisition, analysis and clinical inference), complex (many interactions between operator and patient), and subjective and biased (i.e., having variations from reader).

The underlying technology behind Magnetic Resonance Imaging (MRI) works by applying a strong static magnetic field across a patient in order to excite, align, relax, and measure proton spins to the field. Gradient coils then impose a spatially varying magnetic field across the patient to encode spatial information into the signal received by the MRI system. Finally, strong radiofrequency (RF) coils excite proton nuclei and receive the subsequent signals. The received signal is in the frequency domain (k-space) and needs to be transformed into the image domain to be visually understandable by humans. Images are acquired one line at a time, which limits the potential rate of acquisition. For example, a two-dimensional (2D) 128×128 image requires 128 lines to be acquired at the Nyquist sampling frequency. Likewise, a 128×128×128 three-dimensional (3D) image requires 128×128 lines to be acquired to be at the Nyquist sampling frequency.

Current imaging protocols acquire images in 2D, thereby requiring multiple scans to provide adequate coverage. Although in-plane resolution can be high, the distance between slices is poor, leading to large gaps, which may reduce diagnostic information and bias clinical decision making. Reducing these gaps untenably increases exam times due to both patient discomfort and economic realities. Patients with severe disease who may benefit most from the diagnostic and prognostics of a CMR study often produce the worst quality images due to the above limitations.

Furthermore, the images are acquired during certain times in the cardiac and/or respiratory cycle to minimize physiologic motion artifacts. This requires patient cooperation through timed breath holds, and minimizing bulk motion for the entirety of the scan. This requirement is not always tolerable, particularly for patients with severe diseases. Any images acquired during the wrong windows of the cardiac/respiratory cycle may introduce motion artifacts. Issues like arrhythmias or wall motion abnormalities may impact artifact free visualization of cardiac anatomy/function. These factors individually or combined can result in limited to no diagnostic value and a complete waste of resource that is expensive and in limited supply at the outset of CMR image studies.

Therefore, optimal imaging for each individual patient requires hundreds of decisions from the operator and cooperation of the patient. These decisions, related to scan orientation, registration points, patient anatomy, patient motion, etc. are made while prescribing a complex sequence of diagnostic imaging protocols (e.g., short-axis, long-axis, or 4-chamber, as illustrated in FIG. 1). Given that these decisions are made at the point of care in real time, there are three consequences which impact acquisition in a CMR study: 1) once an image plane is collected during the acquisition phase of the study, it cannot be changed, 2) a high level of expertise is required to correctly and consistently identify the view plane, and 3) identification of the view plane takes a significant amount of time. Improper decisions can lead to non-standard imaging planes and loss of potential diagnostic information or inability to follow standard guidelines. Thus, a CMR study may need to be repeated (resulting in additional appointments, additional time, additional costs) if results are not adequate for a reader to provide a diagnosis.

After the scans are collected, readers (e.g. radiologist, cardiac imaging specialists) are responsible for analyzing and interpreting the study for diagnostic and prognostic purposes. This involves segmenting specific tissue structures to extract various measurements of cardiac morphology and function. This process is time consuming, often taking 40-45 minutes per exam and has a very high rate of inter-reader variability. Variability is exacerbated by the differences encoded into the study from the image acquisition.

The above complexities of CMR all contribute to reduced possible patient access both due to cost and lack of trained operators. Increasing robustness and throughput has the possibility of improving clinical practice.

### BRIEF SUMMARY

According to one example of the present disclosure, an imaging method comprises: causing a magnetic resonance imaging (MRI) system to apply a pulse sequence; acquiring cardiac magnetic resonance (CMR) imaging data of a subject based on the applied pulse sequence; determining a CMR imaging parameter after acquiring the CMR imaging data; and retrospectively reconstructing a CMR image from the acquired CMR imaging data based on the CMR imaging parameter.

In various embodiments of the above example, the CMR imaging parameter is a two-dimensional imaging plane and the CMR image is a two-dimensional cross-sectional image from the imaging plane; the CMR imaging data is continuously acquired for at least 5 minutes and less than 30 minutes; the CMR imaging data is continuously acquired regardless of a cardiac phase or a respiratory phase of the subject, and the method further comprises: automatically identifying the cardiac phase or the respiratory phase of the subject associated with each element of acquired imaging data based on the acquired CMR imaging data, automatically organizing the acquired CMR imaging data according to the identified cardiac phase or the identified respiratory phase, and reconstructing a three-dimensional (3D) time-resolved image from the organized imaging data; the CMR image is retrospectively reconstructed from the 3D time-resolved image; the CMR imaging data is acquired at least once every 10 ms; the 3D time-resolved image is iteratively reconstructed by removing artifact from a plurality of iterations of at least a portion of the 3D time-resolved image; iteratively reconstructing the 3D time-resolved image comprises, for each iteration of the 3D time-resolved image: identifying an artifact in the iteration, determining a stochasticity of the identified artifact, comparing the determined stochasticity to a predetermined threshold, and only removing the identified artifact from the iteration when the determined stochasticity is greater than the predetermined threshold; determining the CMR imaging parameter comprises automatically identifying a short axis, a long axis, or a 4-chamber imaging plane of the 3D time-resolved image, and the CMR image is a two-dimensional, time-resolved cross-sectional image from the 3D time-resolved image along the automatically identified imaging plane; the method further comprises identifying scar tissue by comparing pixels or voxels of portions of the 3D time-resolved image to a predetermined threshold; the 3D time-resolved image has an isotropic resolution less than 2 mm; the cardiac phase or the respiratory phase of the subject is automatically identified based on navigator echo data extracted from the acquired imaging data; the pulse sequence comprises an inversion pulse and the CMR imaging data is continuously acquired for a predetermined period of time following the inversion pulse, and the CMR imaging parameter is an inversion time; the method further comprises: detecting each heart beat of the subject, wherein the inversion pulse is applied in accordance with each detected heart beat; determining the CMR imaging parameter comprises receiving an input from an operator identifying a desired inversion time after the CMR imaging data has been acquired; the predetermined period of time is at least 400 ms; the CMR imaging data is acquired from a thoracic cavity of the subject, extending from at least a neck of the subject to at least a liver of the subject and including an entire heart of the subject; the method further comprises generating a digital twin of the heart of the subject; the CMR imaging data is acquired without a contrast agent in the subject; and/or the pulse sequence is a steady-state free precession (SSFP) sequence.

According to a second example of the present disclosure, an imaging method comprises: during a first acquisition period, causing a magnetic resonance imaging (MRI) system to continuously acquire first imaging data of a subject regardless of a cardiac phase or a respiratory phase of the subject; during a second acquisition period, causing the MRI system to apply a pulse sequence including a heart beat gated inversion pulse, and to continuously acquire second imaging data for a predetermined period of time following the inversion pulse; automatically identifying the cardiac phase or the respiratory phase of the subject associated with each element of the acquired first imaging data based on the acquired first imaging data; automatically organizing the acquired first imaging data according to the identified cardiac phase or the identified respiratory phase; reconstructing a first three-dimensional (3D) time-resolved image from the organized imaging data; determining a desired inversion time after acquiring the second imaging data; and retrospectively reconstructing a second image based on the desired inversion time.

In some embodiments of the second example, a total duration of the first acquisition period and the second acquisition period is at least 5 minutes and less than 30 minutes.

### BRIEF DESCRIPTION OF THE SEVERAL VIEWS OF THE DRAWING

FIG. 1A illustrates an example study protocol according to the present disclosure.

FIG. 1B illustrates an example automated CMR cine method of the present disclosure.

FIG. 2 comparatively illustrates an automated CMR cine acquisition protocol according to the present disclosure and according to conventional techniques.

FIG. 3A illustrates the standard cardiac imaging planes.

FIG. 3B illustrates a comparison of reconstruction according to the standard cardiac imaging planes and the volumetric reconstruction of the present disclosure.

FIG. 4 illustrates an example process for binning and reconstructing MRI data according to the present disclosure.

FIG. 5 illustrates an example iterative image reconstruction process according to the present disclosure.

FIG. 6 illustrates an example system of the present disclosure.

FIG. 7 illustrates an example cursor trace and user interface of a system of the present disclosure.

### DETAILED DESCRIPTION OF THE DRAWING

Based on the foregoing, the present disclosure relates to automated CMR acquisition, reconstruction, and analysis of full three-dimensional (3D) data. According to the present disclosure, almost all interaction between operator and patient, and operator and study, is removed.

In further contrast to current study protocols, which acquire specific orientations and image types one at a time in a two-dimensional (2D) manner (and thereby requiring highly trained operators), the present disclosure relates to CMR protocols that are “always on” 3D semi-random Cartesian acquisitions and that continuously acquires data. Therefore, the systems and methods of the present disclosure produce time-resolved 3D data (herein also referred to as “4D” data), where the number of collected frames represents a temporal resolution. This data constitutes a full “digitized” volume of a patient's thoracic cavity at multiple points in time, rather than discrete slices each at a different point in time.

Low resolution images can be continuously reconstructed throughout the scan to provide real-time troubleshooting. Final diagnostic quality imaging volume can be reconstructed following the end of the study. Accordingly, the present disclosure provides fast volumetric digitization of anatomy & results in high-resolution, orientation-independent views, and measurements with limited user interaction. The results are consistent, easily disseminated, and more easily interpretable since user variability is removed. And study throughput can be increased (and thus order backlogs reduced) with shorter setup, shorter acquisition times, and more consistent study times. Specifically, the acquisition, processing, and analysis of the present disclosure may be instituted by a single input operation of the operator to the system, with the patient simply relaxing for the entirety of the scan—that is, without any human operation or interaction during the scan acquisition, processing, or analysis.

As suggested above, current CMR acquisitions have 4 main limitations:

First, the 2D nature limits diagnostic power and introduces source of diagnostic variability. The “always on” 3D acquisition of the present disclosure solves these problems by providing isotropic (i.e., having the same resolution in all directions; for example, less than 2 mm, and preferably 1.6 mm in a 307 mm field of view) acquisition that covers a large portion of the thoracic cavity, including the heart. In one example, the portion of the thoracic cavity extends from the neck to approximately half of the liver. This reduces inter-scan variability and also provides a more robust scan, thereby reducing the potential need for a second scan.

Second, the high number of decisions requires operator and patient cooperation. The continuous (non-gated) scanning of the present disclosure allows for free-breathing and post-acquisition removal of artifacts due to arrhythmia, bulk motion, and other issues retrospectively. This can reduce scan time, setup time, and improves patient comfort. Further, CMR of the present disclosure is more robust to bulk motion and arrhythmias than conventional techniques.

Third, current scan protocols require a high level of operator expertise. According to the present disclosure, setup is reduced to a single interaction between the operator and patient with minimal impact on scan time or quality. This reduces entry barrier of operator skill, making it more familiar to CT or X-ray technicians. As a result, CMR imaging may be more available in geographies traditionally limited by expensive MRI machines and available clinicians.

Fourth, the low acquisition efficiency leads to patient discomfort, poor quality images, and economic inefficiencies. According to the present disclosure, multiple protocols are coalesced into a single, simple scan (e.g., 20-30 minutes). This reduces complexity and provides more information compared to current protocols, thereby reducing the potential need to bring patients back due to inadequate study quality. The shorter, more consistent acquisition times also enable higher patient throughput. Finally, given the reduced entry barrier, the systems and methods of the present disclosure can enable wider dissemination of CMR techniques, allowing high standards of care to be applied regardless of location or operator.

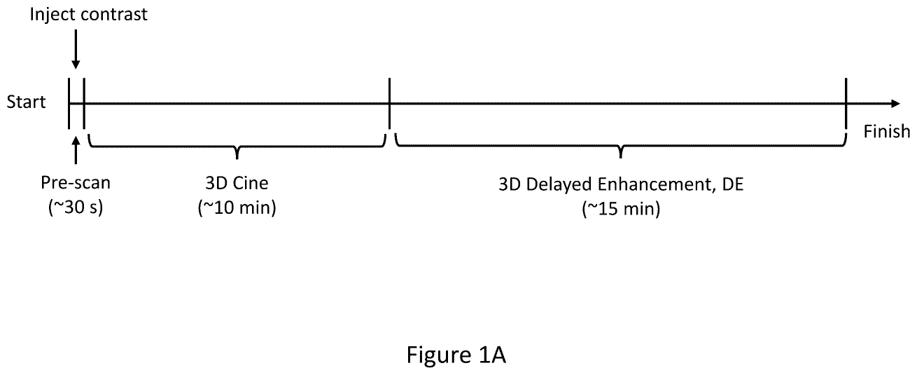

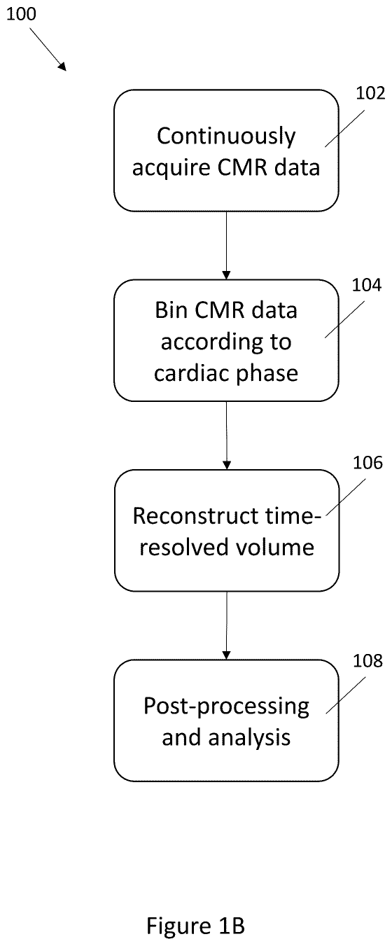

Considering the above, FIG. 1A illustrates an example automated CMR study protocol according to the present disclosure. According to that example, the study begins with an initial short (about 30 second) pre-scan period in which a contrast agent is injected into the patient. This is followed by a cine acquisition period of about 10 minutes and then a delayed enhancement (DE) acquisition period of about 15 minutes. As will be described in more detail below, both the cine and DE acquisition periods can produce 3D image data that can be retrospectively and automatically processed to generate time-resolved images from any plane and/or based on different inversion times, and can be automatically analyzed (e.g., to determine various clinical volume and function metrics). It should be understood that the study protocol of FIG. 1A is merely an example and that the automated CMR techniques of the present disclosure could be applied to study protocols having different time periods for each acquisition protocol, or utilizing only one of the automated CMR cine acquisition or automated DE acquisition. Further, the automated CMR cine acquisition can be performed without a contrast agent.

Depending on the embodiment, these acquisitions can be performed with free-running spoiled gradient echo (GRE) sequences or steady-state free precession (SSFP) sequences. A comparison of an example range of sequence parameters according to the present application (including both automated CMR cine and automated CMR DE), and to traditional cine and DE protocols is shown in Table 1. Data can be collected with Gaussian random Cartesian sampling. Sequences may additionally include navigator echo pulses for a self-gating process (described in more detail below). These sequences of the present disclosure can produce an isotropic resolution of 1.6 mm for a 192×192×192 matrix and a field of view of 307 mm×307 mm×307 mm over 30 cardiac frames (during cine acquisition).

TABLE 1
Comparison of CMR sequence protocols

| | Present | Traditional | Traditional Delayed |
|---|---|---|---|
| | Disclosure | Cine | Enhancement (DE) |
| Flip Angle (FA) | 5°-60° | ≤60° | 10° |
| Repetition | 2.4 ms-4.2 ms | 2.4 ms | 3.7 ms |
| Time (TR) | | | |
| Echo Time (TE) | 1.2 ms-3.1 ms | 1.2 ms | 2.6 ms |
| Resolution | 1.6 mm × 1.6 | 1.5 mm × 1.5 | 1.6 mm × 1.6 |
| | mm × 1.6 mm | mm × 8 mm or | mm × 8 mm |
| | | 1.4 mm × 1.4 | |
| | | mm × 5 mm | |

Particularly regarding the repetition time (TR) and echo time (TE), it is noted that those times may be dependent on available gradient hardware of the MR system acquiring data. For example, for a system with max gradient strength of at least 40 mT/m and max slew of at least 150 T/m/s, the echo time and repetition time may be 2.9 ms and 4.0 ms, respectively, for cine acquisition; and the echo time and repetition time may be 2.6 ms and 3.7 ms, respectively for DE acquisition.

However, more sophisticated systems may allow for reduced echo and repetition times and systems with limited gradient hardware may have increased echo and repetition times. For example, for a system with a max gradient strength of at least 20 mT/m and max slew of at least 100 T/m/s, the echo time and repetition time may be 3.1 ms and 4.2 ms, respectively, for the cine acquisition; and the echo time and repetition time may be 2.9 ms and 4.0 ms for the DE acquisition.

In short, it should be understood that the automated CMR techniques of the present disclosure can be utilized according to any sequence appropriate for given MR system hardware, and can be utilized at any B₀ field strength, for example between 0.055 mT to 9.4 T including 1.5 T, 2.0 T, and 3.0 T. Regardless of the sequence parameters used, the above-described 3D image data that can be retrospectively processed, and having an isotropic resolution of less than 2 mm can be achieved.

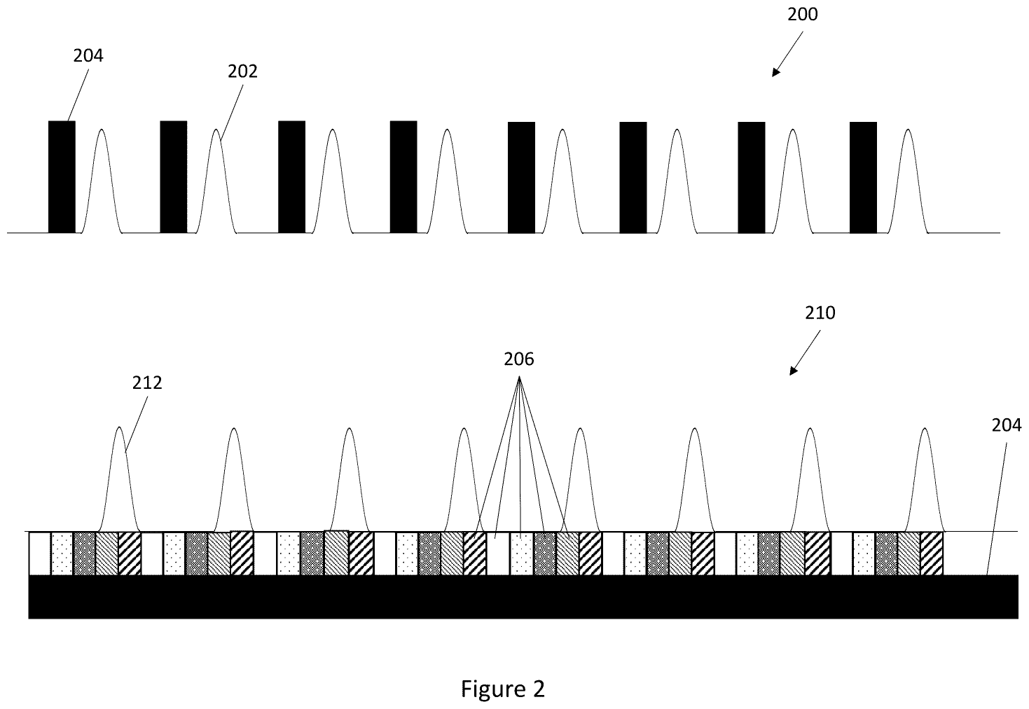

FIG. 1B illustrates a simplified example of an automated CMR cine acquisition method 100 of the present disclosure. As shown therein, the method begins by continuously acquiring CMR data 102 from a CMR imaging device during a scan period (e.g., up to 30 minutes). Herein, “continuous” data acquisition is understood to mean acquisition of data corresponding to a portion of the frequency spectrum of the imaging system on the order of a few milliseconds (e.g., capturing data every 3 ms, or at least once every 5 or 10 ms). This is in contrast to conventional acquisition techniques in which acquisition is gated by the cardiac or respiratory cycle. This distinction is schematically illustrated in FIG. 2, in which a conventional acquisition protocol 200 is illustrated having each acquisition sequence 202 following a gating event 204 (e.g., QRS complex of an ECG signal).

On the other hand, some or all sequences 212 of an acquisition protocol 210 of the present disclosure proceed regardless of the timing relative to the gating event 204 (illustrated as continues for sequences 212), or even in the absence of the gating event 204. Such continuous acquisition is possible, in part, by utilizing signal analysis and/or machine learning methods to identify a portion of the cardiac phase in which the data was acquired without acquiring a separate respiratory or cardiac signal (“self-gating”). As explained in more detail below, the resulting data is then automatically binned (illustrated by various bins 206) based on the identified respiratory or cardiac signal. Further, the patient is permitted to freely breath during the acquisition protocol, and is not required to hold their breath during any portion of the acquisition protocol.

The sampling pattern may be semi-randomized throughout the study, which enables retrospective binning and produces noise-like under-sampling patterns which are addressed with advanced reconstruction techniques. The particular sampling pattern for collecting data may be determined based on a desired spatial resolution, tissue contrast, suppression of imaging artifact, and hardware capabilities. According to one example, the data acquisition is under-sampled (i.e., sampled at a frequency below the Nyquist frequency) in order to reduce scan time. In some embodiments, the particular sampling frequency or a sampling pattern may be at least semi-randomized through at least a portion of the scan. As suggested above, data is preferably acquired for 20-30 minutes (e.g., 25 min) but data acquisition may be possible in as little as 5 minutes. With such a protocol, 2 GB of data may be collected each minute of the scan for a sampling every 3 ms.

As noted above, some acquisition protocols (e.g., DE) may utilize a contrast agent to characterize damaged tissues and perform angiographic imaging and analysis. By injecting a contrasting agent injected into the blood stream, detection of the agent generally corresponds to detection of the blood flow (and thus represents angiographic imaging). And because the contrasting agent can leak into damaged tissue, detection of the agent beyond the blood vessels can be used to identify bad/scarred tissues.

During an automated CMR DE acquisition period, data may be collected with random sampling for a predetermined period of time following application of an inversion pulse. Depending on the embodiment, the inversion pulse is applied on the order of hundreds to thousands of milliseconds. Due to the ‘continuous’ acquisition following the inversion pulse, an operator may retrospectively determine an appropriate or desired inversion time (Ti) (e.g., 50-350 ms) by reconstructing images from the acquired data at that inversion time Ti. In practice, images may be reconstructed for a plurality of inversion times from which a user may select or a user may identify a particular desired inversion time and the image reconstructed based on the identified time. In some embodiments, inversion pulses may be triggered. For example, an inversion pulse may be triggered based on a pulse-oximeter signal or an ECG signal. In these examples, one inversion pulse is provided for each heart beat. Following the inversion pulse, data may be continuously acquired for approximately 400 ms.

Following acquisition, the collected data is binned 102 according to the respiratory and cardiac phase during which the data is collected. This binning can be done automatically by the “self-gating” described above, in which portions of the respiratory and cardiac cycle associated with collected data can be identified by analyzing data collected in response to navigator echo pulses.

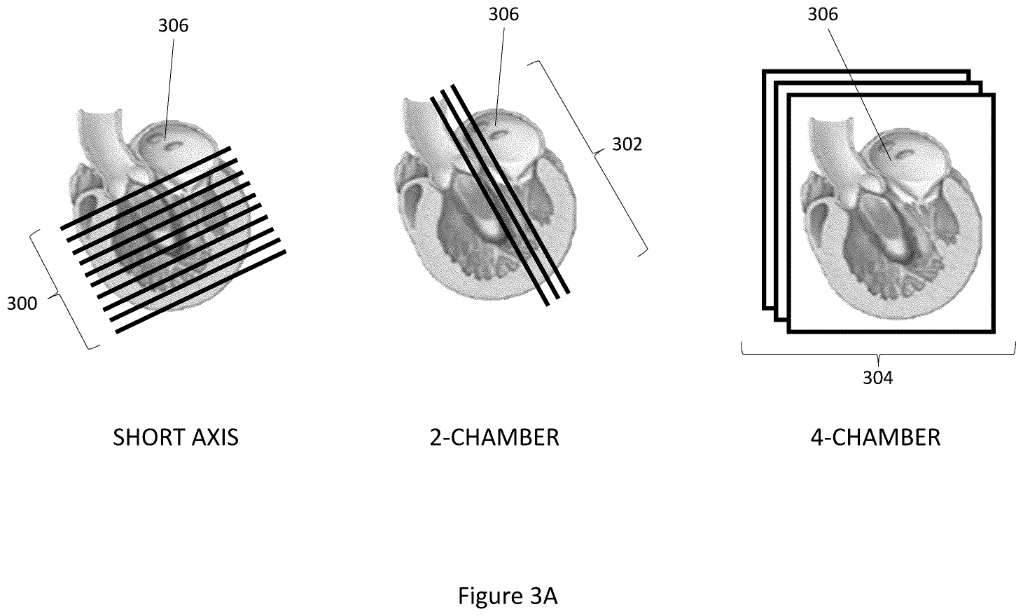

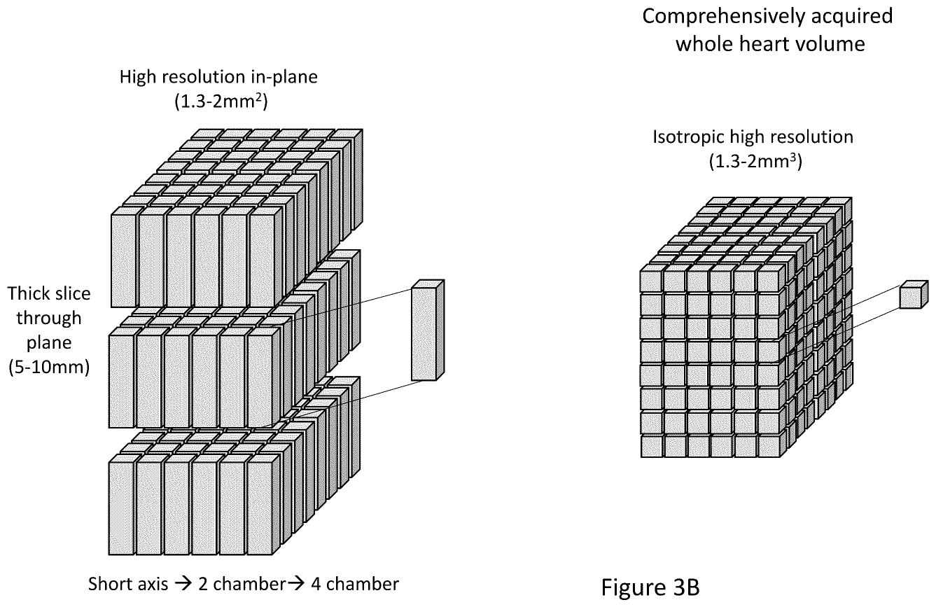

Each bin of data is then reconstructed 106 into a 3D volume and/or one of the standard cardiac imaging planes (e.g., short-axis, 2-chamber, and 4-chamber). FIG. 3A illustrates these planes 300, 302, 304, respectively, relative to a schematic representation of the heart 306. These standard cardiac imaging planes can be automatically identified from the raw and continuously acquired CMR data and/or the reconstructed data.

FIG. 3B illustrates a comparison of acquisition and reconstruction according to the standard cardiac imaging planes, and the volumetric reconstruction of the present disclosure. As shown therein, conventionally images are only available in the slice planes in which they were acquired (different planes requiring separate acquisitions and processing), and with voxels having a different resolution in at least one dimension. In contrast, the present disclosure produces a 3D volume from which 2D images in any plane may be reconstructed. Further, these volumes are produced for multiple points in time (e.g., throughout the cardiac cycle). Thus, the totality of the collected and reconstructed data constitutes time-resolved (4D) data. Additionally, the present disclosure enables real-time low resolution image reconstruction to allow for real-time troubleshooting during the acquisition.

In particular for automated CMR DE acquisition, acquired data from DE scans is time-resolved relative to the inversion pulse rather than a cardiac cycle. In other words, the DE data is binned into different TI times, thereby providing different tissue contrasts. Because the inversion pulse may be triggered by the cardiac cycle as discussed above, the DE data is also time-resolved in terms of the cardiac cycle; however, those times span only a small portion of the cardiac cycle.

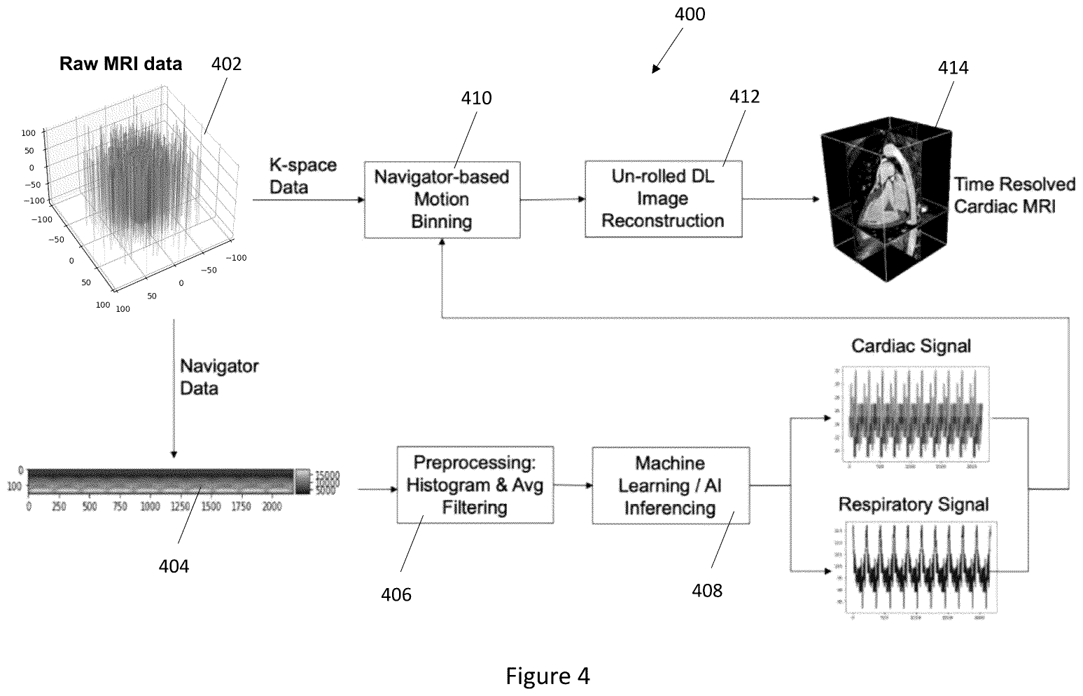

An example process 400 for the binning 104 and reconstruction 106 steps is illustrated in detail in FIG. 4. Preferably, the binning 104 and initial reconstruction 406 occurs as concurrently or immediately after acquisition 102 as possible. In this way, a patient may be re-scanned if necessary (e.g., errors occur during acquisition or data is inadvertently corrupted) during the same visit, thereby reducing call-back appointments.

As seen therein, navigator echo data 404 is first extracted from the raw collected MRI data 402 obtained during acquisition 102. The navigator echo data may then be preprocessed 406, for example, by filtering and input to a machine learning system 408 or other signal/data processing technique. The machine learning system may be trained to identify and output a cardiac and/or respiratory signal from the navigator echo data.

The k-space data from the raw MRI data 402 may then be binned 410 in accordance with the output cardiac and/or respiratory signals. That is, the portions of the k-space data corresponding with a given phase of the cardiac or respiratory cycle (as indicated in the cardiac or respiratory signal) may be grouped together. For example, an R-wave peak may be identified within the cardiac signal and used as a basis for gating and binning the corresponding k-space data.

Finally, k-space data within each bin is reconstructed 412 into 2D or 3D MRI image data 414. The image data from each bin represent images at different times, and thus collectively constitute time-resolved data, or different inversion recovery times which produces different contrast in DE images. In one example, the acquired data has a temporal resolution of at least 25 cardiac frames (and thus 25 bins, and 25 3D volumes). The k-space data may optionally be pre-processed prior to binning 410 and/or reconstruction 412. The previously-mentioned cardiac and/or respiratory signals may be retained for subsequent retrospective image reconstructions.

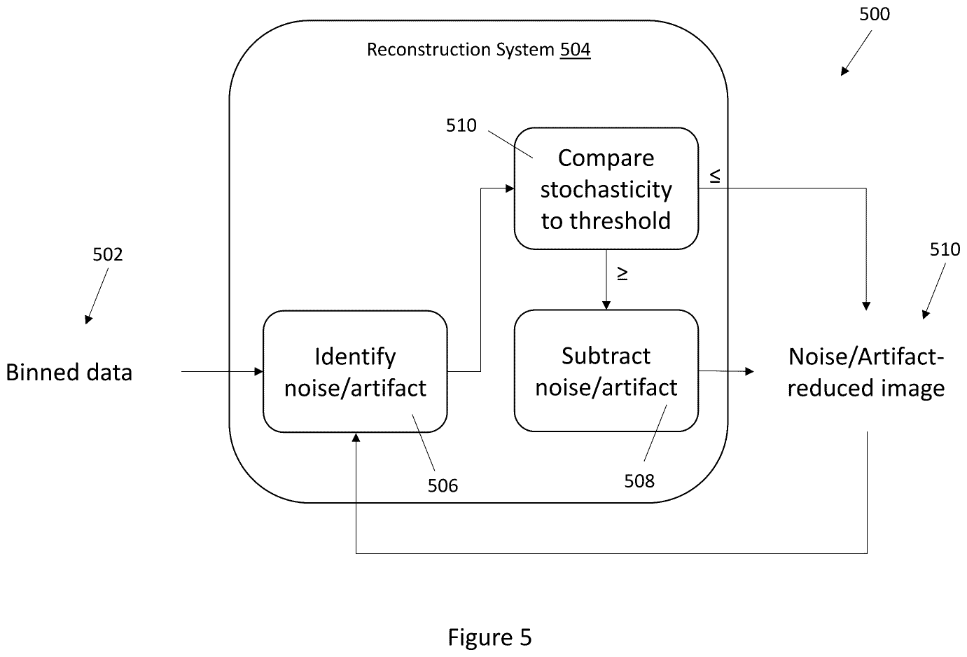

Reconstruction 106 may be performed according to traditional image reconstruction techniques, such as with an inverse Fourier Transform. In some embodiments, the reconstruction may be iterative in a manner that reduces artifacts. An example of an iterative reconstruction process 500 is illustrated in FIG. 5. For example, each iterative reconstruction 504 may be performed at least in part by a machine learning system trained recognize artifacts 506 in each iteration. The recognized artifact is subtracted from the image 508, producing a noise/artifact-reduced image 510, which is then re-input to the trained machine learning system 504. Additionally or alternatively, a low rank denoising technique may be applied during reconstruction.

According to one example, an iterative approach using only the data collected within a single study is used to reduce artifact. Such an approach leverages the reduction of artifact through intelligent combination of coil data (e.g., “SENSE”) and the iterative compressed sensing with total variation regularization (e.g., “CS-TV”). Because typical under-sampling artifact is structured and not random, compressed sensing acquisition protocols can be designed in a manner that makes the under-sampling artifact look more like random noise. This improves the effectiveness of compressed sensing. Machine learning systems can then be trained to identify these types of noise, so that it can be removed. By utilizing these acquisition protocols, the amount of data required to train such a machine learning system can be reduced.

Considering the above, this step can be performed using no additional data besides that acquired during each study at the cost of computational complexity and the potential to introduce motion errors. Once a first set of artifact and/or noise reduced reconstructed images is obtained, that reconstrued data may be used to train a machine learning system to similarly reconstruct image data. The machine learning system may then be iteratively trained, with each subsequent reconstruction. This above training approach has the benefit of being efficient at the point of the study. In some further examples, a series of networks trained to slightly reduce artifact at each stage can be utilized. This “unrolled DL” network (as illustrated in FIG. 4) can be orders of magnitude faster and more efficient than a first stage iterative method. Furthermore, these techniques methods are not mutually exclusive; rather they can be used sequentially to further improve image quality (as measured by higher signal to noise ratio, lower undersampling artifacts, and better robustness to motion).

In some instances, the acquisition pattern produces random, noise-like artifact that further supports the above-described iterative and machine learning reconstruction. By intelligently producing the noise and/or artifact, a machine learning system or other signal processing technique can be designed to accurately detect and remove that noise and/or artifact. For example, because artifact and noise (and other non-structural elements) are more likely to be random, they may be considered highly stochastic. Randomizing the sampling frequency (as suggested above) has the effect of further randomizing aliasing, and thus increasing the stochasticity of the aliasing in each image.

Based on this, in some embodiments the recognized artifact is automatically checked for stochasticity 510 prior to removal. If the stochasticity of the recognized artifact is greater than a pre-determined threshold, it may be determined as appropriate for noise and/or artifact removal 508. However, if the stochasticity is below the pre-determined threshold, that portion of the image may be considered structural and not removed. In such cases, when the iterative reconstruction is re-input to the trained machine learning system 504, the machine may be requested to identify a different portion of the reconstruction as possible artifact. The iterative reconstruction process may be ended when no further artifacts meeting the stochasticity threshold can be recognized. Such a noise/artifact recognition can be performed on an image-by-image or element-by-element (e.g., voxel) basis.

Referring back to FIG. 1B, following reconstruction 106, the time-resolved 3D images may be subject to post-processing and analysis 108 by a clinician or through automated techniques. Depending on the embodiment, the post-processing and/or analysis may include identification of imaging planes and generation of 2D slices, segmentation of anatomical features, creation of digital twins, automated reporting, and/or unlimited retrospective image reconstructions.

In one example, the previously discussed standard imaging planes (or any other plane) can be automatically identified from each reconstructed 3D volume to minimize work for physician interpreters from. This identification can be performed by machine learning systems (e.g., including deep learning models), which are trained to identify cardiac (or other anatomical) landmarks in a way similar to human interpreters. By collecting the raw CMR data according to the present disclosure and producing a whole 3D volume, the view planes can be defined retrospectively. Accordingly, technicians can view any desired two-dimensional (2D) cross-sectional slice of the 3D volumetric image, at any time. Further, retrospective view plane determination can reduce reliance on highly trained operators and reduce setup times. This retrospection also permits infinite plane determination (a la computed tomography) to provide physicians with a powerful tool to evaluate cardiac morphology and function.

Further, the 3D volume and/or individual 2D slices may be segmented to identify anatomical features (e.g., left ventricular myocardium, left ventricular blood pool, right ventricle, left atria, right atria, aorta from the valve to thoracic aorta, plane of aortic valve, and the like). Automated segmentation may be performed by edge detection techniques, machine learning, and the like. Those segmented portions may be further analyzed. For example, analyses may include identifying MR parameters (e.g., T1, T2), image parameters (e.g., contrast, intensity), and/or characterizing, identify biomarkers, and/or identifying physiological parameters of the segmented tissue (e.g., ejection fraction, left ventricular thickness, stroke volume, end systolic volume, end diastolic volume).

Still further, the reconstructed data can be used to generate a complete digital twin of the object being imaged (e.g., the heart). Accordingly, a complete heart model may be generated (e.g., electronically, mathematically, by 3D printing, or the like) that is specific for individual patients. Such models may be used by clinicians to determine appropriate treatments, and plan and rehearse patient-specific operations.

In addition to retrospective generation of 2D slices, images may be reconstructed according to retrospectively determined inversion times. For example, an inversion time Ti=250 ms may be achieved by reconstructing images based on data collected approximately 250 ms after every inversion pulse. In some embodiments, the appropriate or desired inversion time Ti may be selected based on the output of a machine learning system. In other embodiments, the appropriate or desired inversion time Ti may be selected by an operator (e.g., by adjusting a “slider” in a user interface).

In contrast to this retrospective approach, traditional techniques require an operator to pre-select the inversion time, which is used to acquire the original data. And because an improperly selected inversion time may not capture damaged tissues, multiple scans of different inversion times may be required. By retrospectively determining the appropriate or desired inversion time Ti based on the greater duration of data acquisition following each inversion pulse, the present disclosure limits the need for additional scans and additional patient exposure to the contrasting agent. Further, such a feature allows the operator to readily switch between “bright blood” and “dark blood” reconstructions based on a single acquisition. Accordingly, multiple scans are not required to see different types of damaged tissue (e.g., scarring near blood pools where a “bright” depiction of the scar is washed-out by the blood), as with traditional techniques.

Images reconstructed according to the above can still further be automatically analyzed. For example, a region of damaged tissue may be segmented by applying a thresholding technique to the reconstructed images. In other words, the scar tissue may have a higher or lower brightness than regular myocardial tissue in the reconstructed image due to the contrasting agent and inversion pulse time Ti. Thus, by setting an appropriate brightness threshold, the damaged tissue region may be identified within the reconstructed image. The size of the damaged tissue region can then be determined based on the number of pixels/voxels in the identified region (based on a known pixel/voxel size). Additionally or alternatively, the brightness may correspond to a degree of damage and thus a level of damage may be identified based on a brightness level of the damaged tissue region. Additional damaged tissue regions can be identified, and temporal changes of these and other characteristics of any identified damaged tissue can be tracked, by comparing scans of a patient over time (e.g., days, weeks, months, years).

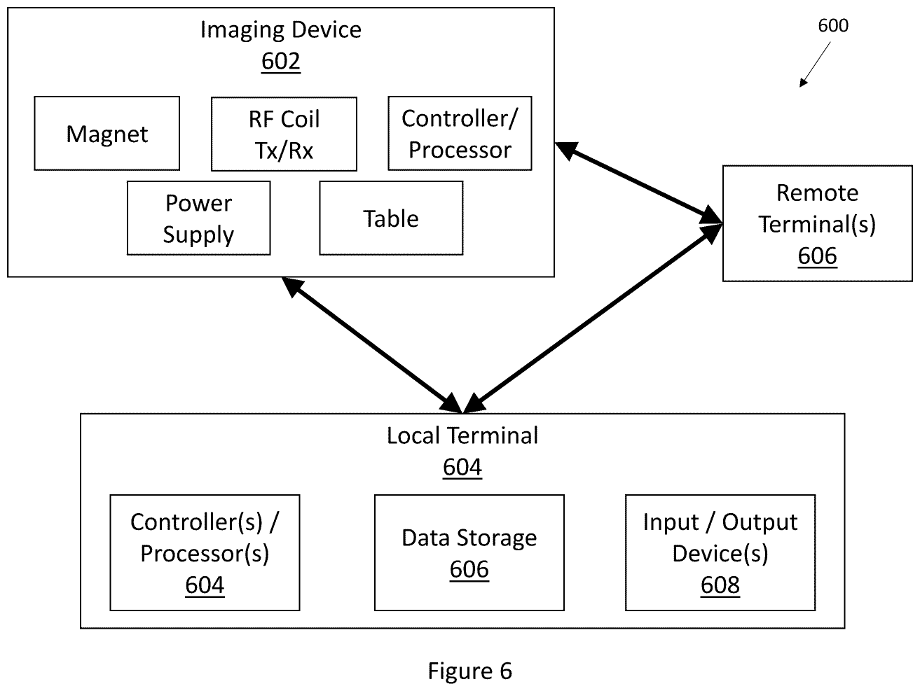

FIG. 6 illustrates an example system of the present disclosure. As shown therein, the system 600 may include the elements of a CMR system. For example, such a system 600 may include an imaging device 602 and a local terminal 604. The local terminal may have one or more controllers/processors 606, data storage 608, and input/output (I/O) devices 610. The imaging device 602 may be a magnetic resonance imaging (MRI) or like system. Such systems may have, for example, a magnet, radio frequency (RF) coil for RF transmission and/or reception, a controller/processor, power supply, patient table, and the like.

The one or more controllers/processors 606 may be collectively configured to control operation of the imaging device according to the above disclosure and/or to process data collected by the imaging device 602. Any one of the controllers/processors 606 may be implemented as a machine learning system, and may comprise integrated circuits, discrete element circuitry, actuators, and the like.

The data storage 608 is configured to store raw data collected by the imaging device 602 and/or data processed by the one or more controllers/processors 606. The data storage 608 may be in any form, such as hard disks, solid state hard drives, flash memory, and the like, and configured to store data in any form such as databases or in data structures, file structures, and the like.

The I/O devices 610 are configured to receive inputs from operators of the system 600 (e.g., to begin data acquisition with the imaging device 602, control desired processing of the data collected from the imaging device 602, and the like), and to output any information associated with the system 600 (e.g., raw data collected from the imaging device 602, reconstructed images, results of analysis of the data collected by the imaging device 602, and the like). Such I/O devices 610 may include touch screens, displays, keyboards, mice, and the like.

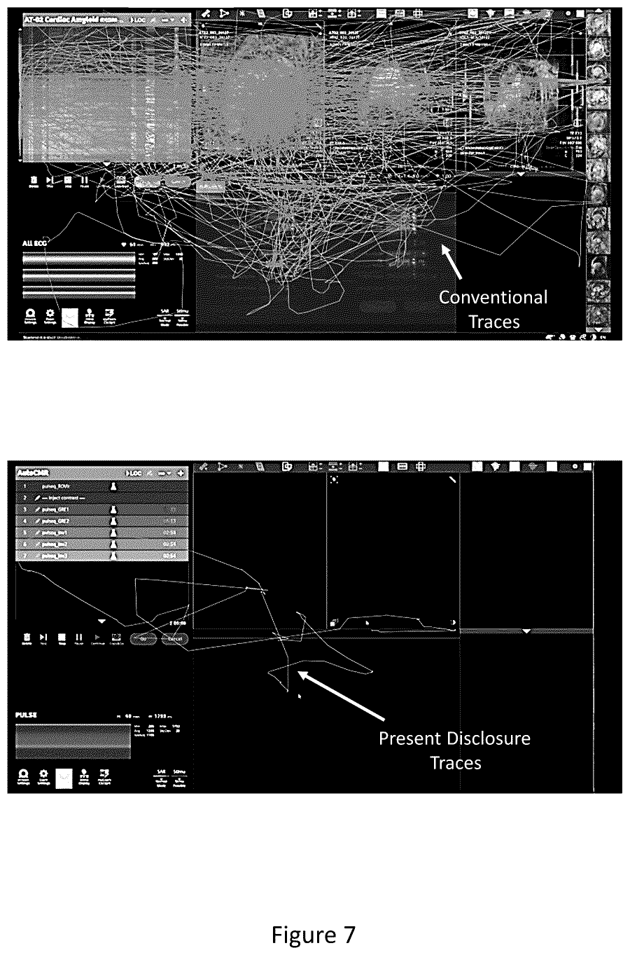

With the present disclosure, user interaction with the I/O devices 610 is greatly simplified and reduced relative to existing technologies. For example, a complete data acquisition protocol can be initiated with as little as a single input, and in some examples no more than ten inputs, from the user. By way of comparison, FIG. 7 illustrates mouse traces over an example user interface for conventional CMR and CMR according to the present disclosure. As can be seen, CMR according to the present disclosure is achieved with significantly less input from the user. Moreover, as described above, the acquired data is significantly more robust.

Any one of the above-described elements of the system may be implemented locally (e.g., as an integrated part of the imaging device 602 or a discrete element of a singular system 600) and/or remotely (e.g., on a server or terminal 620 that is remote from other elements of the system and that communicates with one or more elements in a plurality of systems). For example, data storage may be implemented remotely and accessible by a plurality of systems, or a plurality of terminals of a single system.

In this manner, a patient's data may be available to many imaging systems 600 within a hospital network. In another example, portions of the one or more controllers/processors 606 may be implemented remotely so as to provide a “central command” for controlling all imaging devices within a hospital or hospital network. In this way, a consistent scan protocol can be applied across multiple scanners and disease indications. Similarly, a consistent Cartesian random sampling pattern can be applied across scans, scanners, patients, institutions, and the like. Although particular scan patterns may vary depending on MR hardware, such consistent patterns can be utilized to produce comparable CMR results regardless of location, scanner, time, clinician/technician, and like variables.

In still other examples, existing CMR systems may be modified by upgrading controllers thereof (e.g., by providing a non-transitory computer-readable medium having instructions that cause the controller to operate according to the above-described techniques).

While various features are presented above, it should be understood that the features may be used singly or in any combination thereof. Further, it should be understood that variations and modifications may occur to those skilled in the art to which the claimed examples pertain.

What is claimed is:

1. An imaging method comprising:
causing a magnetic resonance imaging (MRI) system to apply a pulse sequence;
acquiring cardiac magnetic resonance (CMR) imaging data of a subject based on the applied pulse sequence;
determining a CMR imaging parameter after acquiring the CMR imaging data; and
retrospectively reconstructing a CMR image from the acquired CMR imaging data based on the CMR imaging parameter.

2. The imaging method of claim 1, wherein the CMR imaging parameter is a two-dimensional imaging plane and the CMR image is a two-dimensional cross-sectional image from the imaging plane.

3. The imaging method of claim 1, wherein the CMR imaging data is continuously acquired for at least 5 minutes and less than 30 minutes.

4. The imaging method of claim 1,
wherein the CMR imaging data is continuously acquired regardless of a cardiac phase or a respiratory phase of the subject, and
wherein the method further comprises:
automatically identifying the cardiac phase or the respiratory phase of the subject associated with each element of acquired imaging data based on the acquired CMR imaging data;
automatically organizing the acquired CMR imaging data according to the identified cardiac phase or the identified respiratory phase; and
reconstructing a three-dimensional (3D) time-resolved image from the organized imaging data.

5. The imaging method of claim 4, wherein the CMR image is retrospectively reconstructed from the 3D time-resolved image.

6. The imaging method of claim 4, wherein the CMR imaging data is acquired at least once every 10 ms.

7. The imaging method of claim 4, wherein the 3D time-resolved image is iteratively reconstructed by removing artifact from a plurality of iterations of at least a portion of the 3D time-resolved image.

8. The imaging method of claim 7, wherein iteratively reconstructing the 3D time-resolved image comprises, for each iteration of the 3D time-resolved image:
identifying an artifact in the iteration;
determining a stochasticity of the identified artifact;
comparing the determined stochasticity to a predetermined threshold; and
only removing the identified artifact from the iteration when the determined stochasticity is greater than the predetermined threshold.

9. The imaging method of claim 4,
wherein determining the CMR imaging parameter comprises automatically identifying a short axis, a long axis, or a 4-chamber imaging plane of the 3D time-resolved image, and
wherein the CMR image is a two-dimensional, time-resolved cross-sectional image from the 3D time-resolved image along the automatically identified imaging plane.

10. The imaging method of claim 4, further comprising:
identifying scar tissue by comparing pixels or voxels of portions of the 3D time-resolved image to a predetermined threshold.

11. The imaging method of claim 4, wherein the 3D time-resolved image has an isotropic resolution less than 2 mm.

12. The imaging method of claim 4, wherein the cardiac phase or the respiratory phase of the subject is automatically identified based on navigator echo data extracted from the acquired imaging data.

13. The imaging method of claim 1,
wherein the pulse sequence comprises an inversion pulse and the CMR imaging data is continuously acquired for a predetermined period of time following the inversion pulse, and
wherein the CMR imaging parameter is an inversion time.

14. The imaging method of claim 13, further comprising:
detecting each heart beat of the subject,
wherein the inversion pulse is applied in accordance with each detected heart beat.

15. The imaging method of claim 13, wherein determining the CMR imaging parameter comprises receiving an input from an operator identifying a desired inversion time after the CMR imaging data has been acquired.

16. The imaging method of claim 13, wherein the predetermined period of time is at least 400 ms.

17. The imaging method of claim 1, wherein the CMR imaging data is acquired from a thoracic cavity of the subject, extending from at least a neck of the subject to at least a liver of the subject and including an entire heart of the subject.

18. The imaging method of claim 17, further comprising:
generating a digital twin of the heart of the subject.

19. The imaging method of claim 1, wherein the CMR imaging data is acquired without a contrast agent in the subject.

20. The imaging method of claim 1, wherein the pulse sequence is a steady-state free precession (SSFP) sequence.

21. An imaging method comprising:
during a first acquisition period, causing a magnetic resonance imaging (MRI) system to continuously acquire first imaging data of a subject regardless of a cardiac phase or a respiratory phase of the subject;
during a second acquisition period, causing the MRI system to apply a pulse sequence including a heart beat gated inversion pulse, and to continuously acquire second imaging data for a predetermined period of time following the inversion pulse;
automatically identifying the cardiac phase or the respiratory phase of the subject associated with each element of the acquired first imaging data based on the acquired first imaging data;
automatically organizing the acquired first imaging data according to the identified cardiac phase or the identified respiratory phase;
reconstructing a first three-dimensional (3D) time-resolved image from the organized imaging data;
determining a desired inversion time after acquiring the second imaging data; and
retrospectively reconstructing a second image based on the desired inversion time.

22. The imaging method of claim 21, wherein a total duration of the first acquisition period and the second acquisition period is at least 5 minutes and less than 30 minutes.
